(19) United States
(12) Patent Application Publication
(10) Pub. No.: US 2018/0110645 A1
(43) Pub. Date: Apr. 26, 2018

(54) PADDED HEAD POSITIONER OR RESTRAINT

(71) Applicant: Kelly Allen, Mumford, TN (US)

(72) Inventor: Kelly Allen, Mumford, TN (US)

(21) Appl. No.: 15/335,388

(22) Filed: Oct. 26, 2016

(51) Int. Cl. A61F 5/37; A61B 46/20

(52) U.S. Cl. CPC A61F 5/3707; A61B 46/20

(57) ABSTRACT

A padded head restraint (10) is disclosed which includes a head pad (11) having vertical end portions (12) which span a central pad (14). The central pad includes a front surface (15) which is recessed from the front surface (16) of the end walls. The head pad includes a rigid base plate (17), a foam padding (18) overlaying the base plate, and a moisture resistive covering (20) overlaying the base plate and foam padding. A strap (24) is mounted to the head pad. The head positioner includes two oppositely disposed pivotal drape lifts (32).

### TECHNICAL FIELD

This invention relates to a body restraint or positioner, and particularly to a restraint or positioner that is used to restrain the movement of a person's head.

### BACKGROUND OF THE INVENTION

Oftentimes, a medical patient must have his or her head and neck positioned in a particular manner for a medical procedure. For example, in internal jugular (IJ) and external jugular (EJ) vein procedures the head and neck must be turned laterally in order to position the jugular vein for the procedure. The problem is that this positioning of the head and neck is unnatural, thus, a person tends to move the head and neck back to a normal or natural position. This turning of head and neck may result in the interrupted flow of the IV fluids, medications or blood products, crimping of the IV tube, the accidental removal of the IV needle, or a tearing of the patient's skin or vein.

To aid in maintaining a patient's head in the proper position, medical personnel will utilize pillows and/or towels to prop the neck and head. However, this does not restrict the head from moving and the patient's head often reverts back to its natural position because of the softness and lack of rigidity associated with pillows. This turning of the head again may cause the aforementioned problems. Additionally, if the patient is covered with a sterile drape during the procedure, the head movement may cause bleeding and a break of the sterile field, resulting in the costly and time consuming task of reestablishing a sterile field. Lastly, head movement during the insertion of the IV may create a potential danger as the IV needle may stick the medical personnel inserting it.

Prior art head restraints have been designed to immobilize a patient's head. However, these restraints are typically made of a rigid or hard plastic to prevent head movement. These restraints are extremely uncomfortable for the patient.

Accordingly, it is seen that a need remains for a head positioner and restraint to restrict head movement during a medical procedure. It is to the provision of such therefore that the present invention is primarily directed.

### SUMMARY OF THE INVENTION

In a preferred form of the invention a padded head positioner comprises a head pad having two oppositely disposed vertical end portions of a first select height and a central portion extending between the two vertical end portions having a second selected height less than the first selected height of the two end portions. The padded head positioner also includes a flexible strap coupled to each end portion and extending across the central portion. The strap is adjustable in length across the head pad.

### BRIEF DESCRIPTION OF THE DRAWING

FIG. 1 is a perspective view of a head positioner embodying principles of the invention in a preferred form, shown coupled to a person's head.

FIG. 2 is a perspective view of a head positioner in another embodiment.

### DETAILED DESCRIPTION

With reference next to the drawings, there is shown a head positioner or restraint 10 in a preferred form of the invention. The head positioner 10 includes a resilient head pad 11 having a pair of vertical end portions, ends, end walls, end pads, or bolsters 12 which are spanned by a central portion, central pad, or bridge 14. The central pad 14 includes a front surface 15 which is recessed from the front surface 16 of the end walls 12. The end walls 12 and central pad 14 are soft or cushioned for a person's comfort while firm enough to restrict head movement under a person's normal physical exertion.

The head pad 11 includes a firm or rigid base plate 17 upon which is coupled a resilient or compressible foam padding 18. The base plate 17 includes a bottom portion 17′ and two oppositely disposed side portions 17″ which extend upwardly from the bottom portion 17′. The base plate 17 and padding 18 are overlaid with a moisture resistive covering 20. The base plate 17 may be made of any suitable firm material, such as wood, plastic, metal or the like. The padding 18 may be made of a compressible material such as an open-celled or closed-celled foam. The covering 20 is preferably made of a moisture resistive material, such as vinyl, but may also be made of other materials such as leather, fabric or the like. The covering material is preferably a color which contrasts with a person's body and body fluids so that medical personal may easily detect a problem, such as bleeding from an IV site.

An elongated, flexible restraint or strap 24 is mounted to the head pad 11. The strap 24 includes two strap anchors 26 mounted to the outer sidewall 25 of the vertical walls 12, although it should be understood that one end of the strap may be mounted or coupled directly to the cover without the need of an anchor. One end of the strap 24 is permanently coupled to one strap anchor 26. The opposite or free end of the strap 24 passes through the second anchor 26 and is coupled to itself through hook and loop type fasteners 27 mounted to the strap, or other type of known fastener. The free passage of the strap 24 through the second strap anchor 26 allows the length of the strap between the two strap anchors to be varied, so that the strap is adjustable with respect to a person's head positioned upon the head positioner. It should be understood that the strap may be in any conventional form, such as a two piece strap having mating male and female releasable buckles or fasteners.

Lastly, the head positioner 10 includes two oppositely disposed drape lifts 32. Each drape lift 32 includes a body portion 33 and a mounting screw 34 extending through a bottom part of the body portion 33 to allow pivotal motion of the body portion relative to the head pad 11. Each drape lift 32 is pivotally mounted to one outer sidewall 25 of the vertical end walls 12. The drape lift 32 may be pivoted between a stowed position stored within the periphery of the outer sidewall 25, and an in-use position oriented generally vertically so that a large portion of the drape lift 32 extends beyond the top edge of the periphery of the outer sidewall 25. The drape lift 32 may be made of any material, but is preferably a non-absorbent material such as plastic or metal.

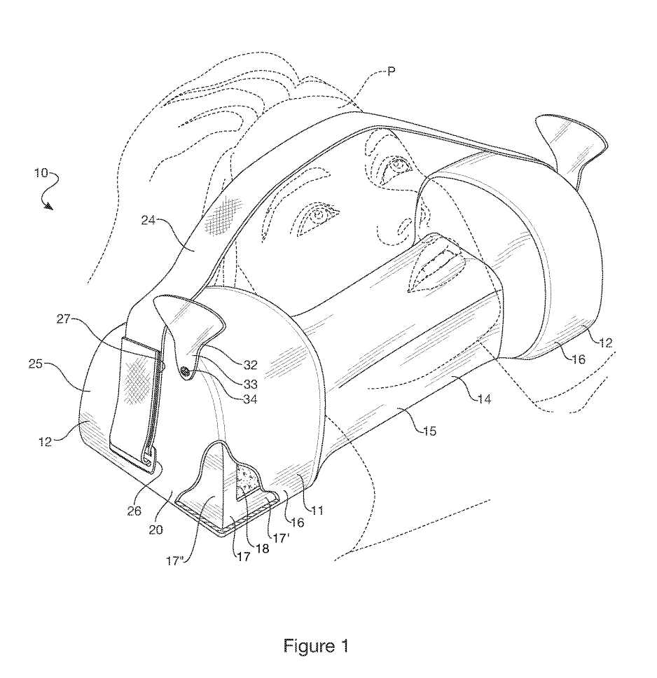

In use, the head position 10 is placed upon a support surface in an upright orientation shown in FIG. 1. A person's head is then positioned upon the central pad 14. The positioning of the head and neck depends upon the procedure being conducted and the body access desired.

Once the head is properly positioned, the strap 24 is positioned over the person's head and tightened to immobilize the head. The strap 24 is maintained in its tight configuration through the mating of the hook and loop fasteners 27. The head is prevented from turning or rotating through the vertical end walls 12 positioned on either side of the head. With the head now in a stable condition, the medical personnel may proceed with the medical treatment or procedure.

Should a medical drape which covers the patient's head P (shown in phantom lines) be utilized during the medical procedure, the drape lifts 32 are pivoted to their in-use position, shown in FIG. 1. While the patient's head is shown in a generally upright position (looking upwardly), it should be understood that with most procedures utilizing the position the patient's head is turned to the side relative to the body so that the jugular vein is exposed. With the drape lifts 32 now position above the height of the head pad 11, the drape is maintained at a position spaced from the patient's face. This positioning and maintenance of the drape away from a patient's face allows a patient to breath normally while also minimizing any claustrophobic or confined feelings a patient may have when confronted with a drape resting upon the face.

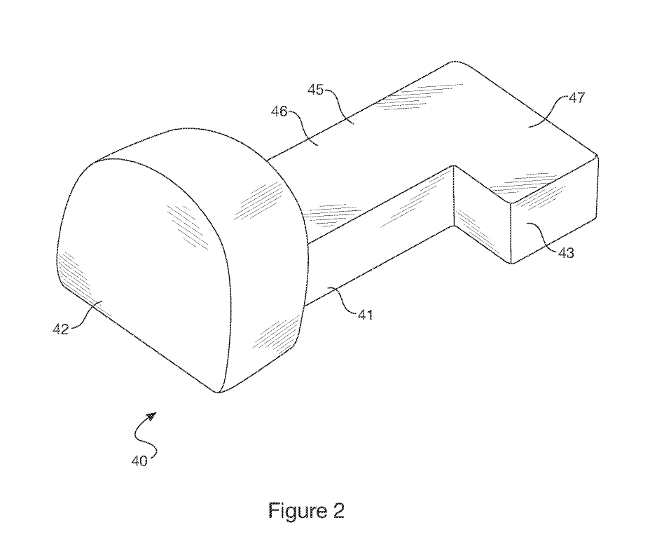

FIG. 2 shows a head restraint 40 in another preferred form of the invention. The head restraint is essentially the same as that previously referenced with regard to FIG. 1, except for the shape of the head pad 41. Here, the head pad 41 has a high, stationary vertical end wall 42 on one end and a low vertical end wall 43 on the opposite end, the low vertical end wall 43 may also be thought of as simply an extension or part of a central portion 45. This type of configuration may be better suited for medical procedures wherein the head is turned to one side, i.e., wherein the patient is facing the low vertical end wall 43.

The central portion 45 includes a leg portion 46 extending horizontally and generally normally from the end wall 42 and a foot portion 47 extending horizontally and generally normally from the leg portion 46. The foot portion 47 may be considered the low vertical end wall 43. Thus, the central portion 45 is generally L-shaped or J-shaped in a horizontally orientation. The foot portion 47 aids in providing a wider stance to increase stability of the head restraint.

It should be understood that the base plate 17 provides rigidity to the head pad to insure that the head is maintained in a proper position for the medical procedure, yet, the padding 18 provides some softness to insure comfort during the medical procedure. The base plate 17 also prevent the rolling of the head position as the rigid nature prevents folding of the head pad 11. Also, the padding 18 is soft enough to be comfortable but firm enough to maintain the proper head position.

It thus is seen that a head restraint is now provided which overcomes problems associated with those of the prior art. While this invention has been described in detail with particular references to the preferred embodiments thereof, it should be understood that many modifications, additions and deletions, in addition to those expressly recited, may be made thereto without departure from the spirit and scope of the invention as set forth in the following claims.

1. A padded head positioner comprising,
a resilient head pad having two oppositely disposed vertical end portions of a first select height and a central portion extending between said two vertical end portions having a second selected height less than said first selected height of said two end portions, and
a flexible strap coupled to each said end portion and extending across said central portion, said strap being adjustable in length across said head pad.

2. The padded head position of claim 1 further comprising at least one drape lift coupled to said head pad.

3. The padded head positioner of claim 2 wherein said at least one drape lift is pivotal between a stowed position and an in-use position.

4. The padded head positioner of claim 3 wherein when said at least one drape lift is in its in-use position, the drape lift extends above the vertical end portion.

5. The padded head positioner of claim 1 wherein said head pad includes a rigid base plate, a padding overlaying said base plate, and a fluid impervious cover covering said padding.

6. The padded head positioner of claim 1 wherein said central portion has a front surface which is inwardly recessed from a front surface of said end portions.

7. The padded head positioner of claim 6 wherein said rigid base plate includes a bottom portion and two oppositely disposed side portions extending upwardly from said bottom portion.

8. A padded head positioner comprising,
a head pad having a stationary vertical end portion of a first select height and a central portion extending from said stationary vertical end portion having a second selected height less than said first selected height of said stationary vertical end portion, said head pad having a rigid base plate, a resilient pad positioned upon said rigid base plate, and a cover overlaying said resilient pad.

9. The padded head positioner of claim 8 wherein said rigid base plate includes a bottom portion and two oppositely disposed side portions extending upwardly from said bottom portion.

10. The head positioner of claim 8 wherein said cover is a fluid impervious cover.

11. The head positioner of claim 8 wherein said central portion has a front surface which is inwardly recessed from a front surface of said end portion.

12. The head positioner of claim 8 wherein said central portion includes an elongated leg portion extending horizontally from said stationary vertical end portion and foot portion extending horizontally from said leg portion.

13. The head positioner of claim 8 wherein said central portion is generally L-shaped.
